(19) United States
(12) Patent Application Publication
(10) Pub. No.: US 2023/0183662 A1
(43) Pub. Date: Jun. 15, 2023

(54) NOVEL DNA METHYLTRANSFERASE

(71) Applicant: Zhejiang University, Hangzhou (CN)

(72) Inventors: Yuejin Hua, Hangzhou (CN); Shengjie Li, Hangzhou (CN); Liangyan Wang, Hangzhou (CN); Jianling Cai, Hangzhou (CN); Xiaoting Hua, Hangzhou (CN)

(21) Appl. No.: 17/552,076

(22) Filed: Dec. 15, 2021

(51) Int. Cl. C12N 9/10

(52) U.S. Cl. CPC C12N 9/1007; C12Y 201/01037

(57) ABSTRACT

A DNA methyltransferase is derived from Deinococcus radiodurans and has a typical conservative structural domain of DNA methyltransferase. The DNA methyltransferase includes: an AdoMet binding region containing a “FxGxG” conservative sequence, a target sequence recognition region and a catalytic region containing a “TSPPY” conservative sequence sequentially from N-terminal to C-terminal; and belongs to α-type DNA methyltransferase category. The recognized substrate DNA conservative sequence is 5′-CCGCGG-3′, a methylation modified position is N4 site of second cytosine to generate a 4mC type modified base, and an optimum temperature for methylated reaction is in a range of 25-37° C. The DNA methyltransferase can specific-recognize the conservative motif of “CCGCGG” and methylate the N4 site of the second cytosine to produce the 4mC modified base, which is a N4-Cytosine DNA methyltransferase.

### TECHNICAL FIELD

The invention relates to a deoxyribonucleic acid (DNA) methyltransferase M.DraR1 of Deinococcus radiodurans, and more particularly to characteristics of a DNA conservative sequence recognized by the DNA methyltransferase and a methylation modification mode thereof.

### STATEMENT REGARDING SEQUENCE LISTING

The sequence listing associated with this application is provided in text format in lieu of a paper copy and is hereby incorporated by reference into the specification. The name of the text file containing the sequence listing is updated_sequence_listing. The text file is 18853 bytes; was created on Apr. 6, 2023; contains no new matter; and is being submitted electronically via EFS-Web.

### BACKGROUND

DNA modification can cause changes in chromatin structure, and DNA conformation and stability, leading to the change of DNA interacting with protein, which expands a structural complexity and information depth of DNA while regulating gene expression. Apparent information transmitted by chemical modifications of four bases exist in all living organisms including viruses and bacteriophages. DNA methylation modification is a widespread and very important DNA epigenetic modification, which has an important regulatory effect on growth, maintenance of genome stability and differentiation of organisms.

Enzymes that mediate and catalyze DNA methylation modification are mainly DNA methyltransferases, which are divided into three categories according to different products formed. Two categories of the enzymes act on amino groups outside base rings to produce m-6A and m-4C methylation modifications, and the other category of the enzymes catalyzes and produces m-5C. The number of methylases and recognized conservative motifs are widely variable among different species of bacteria, and even in closely related strains, while the number of enzymes and recognition sites are not the same. DNA methyltransferases have been widely used in important fields such as genetic engineering, molecular biology experiments and even drug targets for bacterial infections. The development and utilization of new DNA methyltransferases is of great significance to life science research.

Deinococcus radiodurans has a strong ability to repair DNA damage, and is highly resistant to mutations and lethal effects caused by ionizing radiation, ultraviolet rays, drying, and various DNA damage chemical reagents. The strong anti-radiation ability of the bacterium benefits from its various characteristics, such as having some new genes or new functions that are different from other organisms. A product of DNA methyltransferase M.DraR1 expressed by in vivo M.DraR1 gene (gene name is dr_C0020, protein sequence ID is ANC73351.1) is composed of 434 amino acids and has a typical DNA methyltransferase conservative structural domain, and belongs to α-type (alpha-type) DNA methyltransferases. However, a specific modification mode and a substrate recognition sequence of M.DraR1 have not been reported.

### SUMMARY

In order to overcome the shortcomings of the prior art, an objective of the invention is to provide a DNA methyltransferase of Deinococcus radiodurans.

Specifically, a DNA methyltransferase of Deinococcus radiodurans includes: an Adenosylmethionine (AdoMet) binding region containing a “FxGxG” (where “x” is a wildcard symbol representing at least one letter) conservative sequence, a target sequence recognition region and a catalytic region containing a “TSPPY” conservative sequence shown in SEQ ID NO: 7, sequentially arranged in that order from N-terminal to C-terminal; and belongs to α-type DNA methyltransferase category/group. An amino acid sequence of the DNA methyltransferase is shown in SEQ ID NO: 1, or an amino acid sequence formed by the amino acid sequence shown in SEQ ID NO: 1 after substitution, deletion or addition of one or more amino acids and having DNA methyltransferase activity. The DNA methyltransferase is capable of recognizing a DNA conservative sequence of 5′-CCGCGG-3′, and methylation modifying N4 site of second cytosine (C) thereof to generate a 4mC type modified base.

In an embodiment, a reaction buffer for the DNA methyltransferase contains 50-200 millimoles (mM) potassium chloride (KCl), 10-50 mM trimethylolaminomethane hydrochloride (Tris-HCl) with pH7.5-8.0, 0.1 mM ethylenediaminetetraacetic acid (EDTA), 3-7 mM 13-Mercaptoethanol 03-Me), and 20-100 micromoles (μM) S-adenosyl methionine (SAM).

In an embodiment, an enzyme activity temperature range of the DNA methyltransferase is 4-60 Celsius degrees (° C.).

In an embodiment, an optimum enzyme activity temperature range of the DNA methyltransferase is 25-37° C.

Beneficial effects can be achieved by the invention are as follows:

the invention provides an unreported DNA methyltransferase and its recognized conservative sequence and methylation modification type, which can provide basic data for the development and application of new restriction-modification enzymes, and is of great significance for the development of new tool enzyme in molecular biology.

### BRIEF DESCRIPTION OF DRAWINGS

FIGS. 1A and 1B show a construction principle of M.DraR1 gene knockout strain and polymerase chain reaction (PCR) identification results of a DNA methyltransferase according to the invention. In which, FIG. 1A is a schematic diagram of knockout principle, and FIG. 1B is the PCR identification results; lanes 1, 3 and 5 are amplification results of primers P1P4; lanes 2, 4 and 6 are internal verification results of primers P5P6; the lanes 1 and 2 are wild-type DraR1 control groups, the lanes 3 and 4 are test groups of the M.DraR1 gene knockout strains cultured in TS media, and the lanes 5 and 6 are test groups of the M.DraR1 gene knockout strains cultured in TGY media.

FIG. 2 is a representative diagram of a 4mC methylation modified “CCGCGG” motif of a wild-type genomic DNA of Deinococcus radiodurans after three-generation single-molecule sequencing analysis, but 4mC methylation modification was not detected in the same “CCGCGG” motif in the genome of M.DraR1 gene knockout strain, showing that a conservative motif recognized by M.DraR1 is “CCGCGG”.

FIG. 3 shows contents of 4mC modifications in genomes of the wild-type DraR1 and M.DraR1 gene knockout strain of Deinococcus radiodurans, expressed by m4dC/dC ratio in permillage (‰).

FIG. 4A and FIG. 4B are schematic diagrams of a conservative structural domain of DNA methyltransferase M.DraR1, where the M.DraR1 has a typical conservative structural domain, specifically from N-terminal to C-terminal, there are a AdoMet binding region containing “FxGxG” conservative sequence, a target sequence recognition region (TRD sequence) and a catalytic region containing “TSPPY” conservative sequence shown in SEQ ID NO: 7 sequentially arranged in that order, and belongs to α-type N4-Cytosine DNA methyltransferase category. In addition, in FIG. 4B, conservative amino acid residues, relatively conservative residues, and non-conservative residues are denoted by letters with different colors and backgrounds.

FIGS. 5A-5C are schematic diagrams of in vivo methylated enzyme activity analysis of DNA methyltransferase M. DraR1. In which, FIGS. 5A and 5B are schematic diagrams of digestion positions of pRRS-M.DraR1 plasmid through restriction endonucleases of Hind III and Sac II, and FIG. 5C shows agarose electrophoresis results of the pRRS-M.DraR1 plasmid and its PCR product after being digested by the hind III and sac II, Lanes 1 and 3 are effects of the PCR product of pRRS-M.DraR1 plasmid and the plasmid itself without being digested, and Lanes 2 and 4 are results after being digested.

FIG. 6 is a schematic diagram of in vitro methylated enzyme activity analysis of DNA methyltransferase M.DraR1. In which, Lane 1 is an original substrate control group, i.e., being not digested with Sac II; Lane 2 is unmethylated negative control group, i.e., being not methylation modified by target protein but being digested with Sac II; and Lanes 3, 4 and 5 are positive results of being methylation modified under reaction conditions of 25° C., 30° C. and 37° C. and digested with Sac II, respectively.

### DETAILED DESCRIPTION OF EMBODIMENTS

The invention will be further described below in conjunction with the drawings and specific embodiments.

### Embodiment 1: Substrate Recognition Specificity of DNA Methyltransferase M.DraR1 of Deinococcus radiodurans

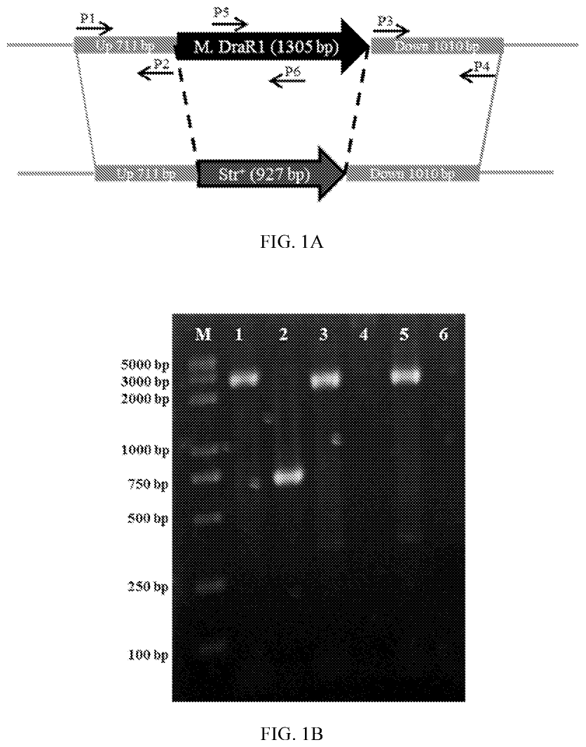

(1) Construction of M.DraR1 gene deletion/knockout strain of DNA methyltransferase: a classic “triple-segment ligation method” is used, an upstream segment (about 710 bp) and a downstream segment (about 1000 bp) of the M.DraR1 gene sequence and a resistance gene segment (streptomycin resistance gene, shorted as Str, about 927 bp) are linked into a whole segment (see FIG. 1A) in vitro by T4 ligase (Takara Company), and then the triple-segment product is transformed into a competent cell of Deinococcus radiodurans, mutant strains then are screened on a TGY culture medium plate containing the Str resistance, and the M.DraR1 gene deletion strain −ΔM.DraR1 (see FIG. 1B) is obtained after PCR and sequencing verification.

(2) Extraction of genomes of the wild-type DraR1 and M.DraR1 gene knockout strain of Deinococcus radiodurans: single colonies of the wild-type DraR1 and the knockout strain 4M.DraR1 are selected and put into 5 mL TGY (TGY containing Str antibiotics at a final concentration of 10 μg/mL) and 5 mL TS liquid culture media respectively, and cultured for 35-40 h at a vibration of 220 rmp and a temperature of 28±5° C.; afterwards, each of them is taken with 50 μL and put into 50 mL TGY and 50 mL TS liquid culture media respectively, and are cultured under the same culture conditions until O.D.₆₀₀ is within the range of 1.0-3.0, bacteria/cells are then collected by centrifugation at 12000 rpm, and genomic DNAs are extracted as per operations of bacterial genomic DNA Extraction kit (Tiangen Bio Company).

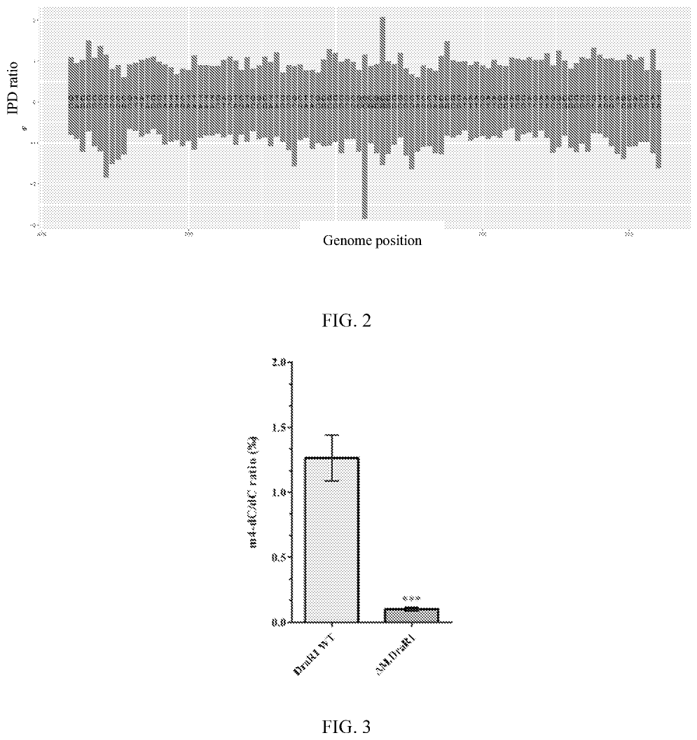

(3) PacBio (Pacific Biosciences) three-generation sequencing (single-molecule real-time technology, SMRT): DNA samples qualified by electrophoresis detection are broken into target segments with a size required for library construction by Covaris g-TUBE, after DNA damage and terminal repairs, a hairpin adapter is linked to both ends of each the DNA segment using DNA cohesive enzymes, AMpure PB magnetic beads then are used for purification and selection of the DNA segments to thereby construct a SMRT Bell library. After the purified segments are re-dissolved in buffer, BluePipin segments are used to screen segments with a specific size for further magnetic bead purification. The constructed library is quantified by Qubit concentration, Agilent 2100 is used to detect sizes of inserted segments, and then the PacBio RSII platform is used for sequencing analysis. The result shows that a recognizable DNA conservative sequence of the DNA methyltransferase M.DraR1 is 5′-CCGCGG-3′, and it can be methylation modified at N4 site on the second cytosine (C) to produce a 4mC type modified base (See FIG. 2 and Table 1). Table 1 is a statistical table of methylation status of “CCGCGG” DNA conservative motifs in genomes of the wild-type DraR1 and the M.DraR1 gene knockout strain of Deinococcus radiodurans.

TABLE 1

| | total number of | total number and ratio of |
|---|---|---|
| | “CCGCGG” motif in | methylation modified “CCGCGG” |
| Strain | genome | motif |
| DraR1 WT | 696 | 667 (96%) |
| ΔM.DraR1 | 696 | 0 |

Ultra high performance

liquid chromatography triple quadrupole tandem mass spectrometry (UHPLC-QQQ-MS/MS) analysis: 2-4 μg of genomic DNAs are dissolved into nuclease-free water, an ice bath then is taken immediately after high temperature denaturation, and subsequently nuclease P1, phosphodiesterase I and alkaline phosphatase are used sequentially in that order for full enzymatic hydrolysis, the sample after enzymatic hydrolysis into single nucleoside is diluted 2 times and filtered, and several microliters of the sample is taken for analysis. Standard curves are used to quantitatively analyze different single nucleosides and then ratios in permillage of modified bases to standard bases are calculated. The result shows that the M.DraR1 methylase is N4-Cytosine DNA methyltransferase (see FIG. 3).

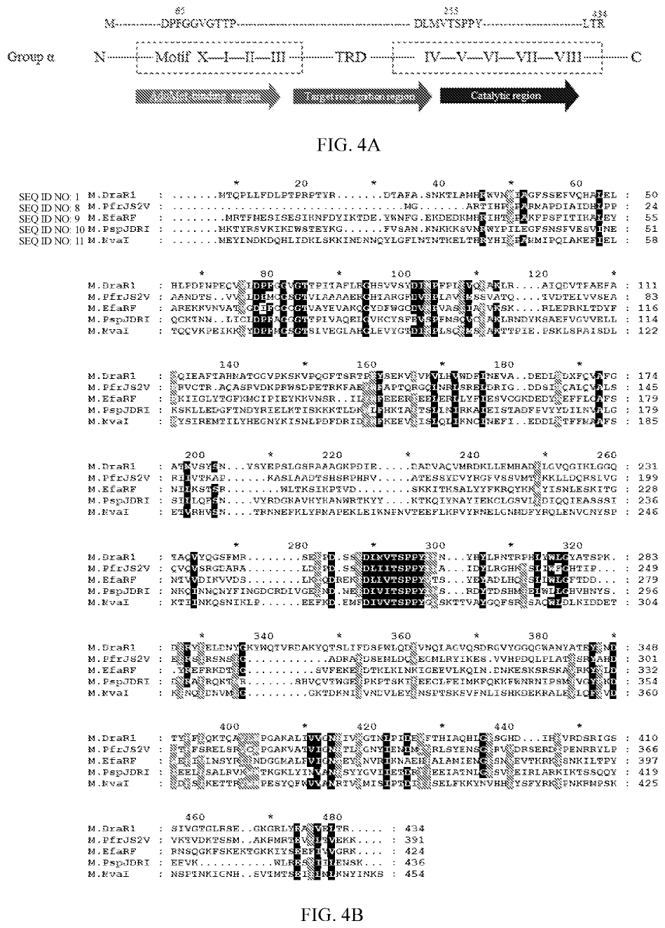

(5) Bioinformatics analysis of DNA methyltransferase M.DraR1: The CLC Sequence Viewer 7 software is used to compare the amino acid sequence in SEQ ID NO: 1 with other N4-Cytosine DNA methyltransferase protein sequences, and the result further indicates that the M.DraR1 methylase is the N4-Cytosine DNA methyltransferase (see FIGS. 4A and 4B). The recognizable DNA conservative sequence by the M.DraR1 methylase is “CCGCGG” and different from conservative sequences recognizable by other N4-Cytosine DNA methyltransferases, and it is indicated that the DNA methyltransferase encoded by this gene is a new type of protease, which may contribute to the specific resistance mechanism of Deinococcus radiodurans (see Table 2). Table 2 is a statistical chart showing the number of amino acids of N4-Cytosine DNA methyltransferase selected for bioinformatics analysis, recognized conservative sequences and modified positions, protein IDs, and species information.

TABLE 2

| | Number of | Recognized | | |
|---|---|---|---|---|
| Name | amino acids | target motif | Protein ID | Latin name of strain |
| M.DraR1 | 434 aa | C^{4m}CGCGG | ANC73351.1 | Deinococcus radiodurans R1 |
| M.MvaI | 454 aa | C^{4m}CWGG | CAA34854.1 | Micrococcus varians RFL19 |
| M.EfaRFII | 424 aa | C^{4m}CGG | AEA94510.1 | Enterococcus faecalis OG1RF |
| M.PspJDRI | 436 aa | ^{4m}CCGG | ACS98972.1 | Paenibacillus species JDR-2 |
| M.PfrJS2V | 391 aa | GC^{4m}CGGC | SBM44409.1 | Propionibacteriu freudenreichii JS2 |

### Embodiment 2: In Vivo Enzyme Activity Analysis of DNA Methyltransferase M.DraR1

(1) Construction of in vivo expression vector namely pRRS-M.DraR1 vector: genomic DNA of Deinococcus radiodurans is used as a template, and the M.DraR1 gene is amplified by in vitro PCR with pfu high-fidelity polymerase (full-form gene, AP 221). An upstream primer pRRS-M.DraR1-F contains Sbf I digestion site and TTAAGG box and TTAATCAT sequence, and a sequence thereof is shown in SEQ ID NO:2. A downstream primer pRRS-M.DraR1-R contains BamH I digestion site and CCGCGG substrate conservative sequence, and a sequence thereof is shown in SEQ ID NO: 3. 1.0% agarose electrophoresis detection is carried out, the PCR product is purified (by DNA purification kit of Life company, Cat NO. 116401), purified PCR segment and pRRS plasmid (gifted by Professor Roberts from NEB company) are simultaneously digested by use of Sbf I and BamH I (NEB company), the digested segments then are purified and ligated with T4 DNA ligase, and the ligated products are transferred into E. coli ER2796 competent cells and the method can refer to the transformation step in the “Molecular Cloning Guide”. Clones are selected from a LA solid culture dish (a final concentration of 100 μg/mL ampicillin Amp is added to LA culture medium) for sequencing identification (the sequence of the upstream sequencing primer pRRS-F is shown in SEQ ID NO: 4, and the downstream sequencing primer pRRS-R is shown in SEQ ID NO: 5), and if the sequencing result is correct, it is regaded as a positive clone.

(2) Extraction of pRRS-M.DraR1 plasmid: positive clones are selected and put into 5 ml LA liquid culture medium, and then are cultured overnight at 37° C. and 220 rpm of vibration; afterwards, 50 μL thereof is absorbed/picked and transferred into 50 ml LA liquid medium, cultured for 20±2 h under the same culture conditions, bacteria are collected by centrifugation at 12000 rpm, and then the plasmid is extracted by a plasmid extraction kit (DNA American AxyGen company, Cat. No. AP-MN-P-250G).

(3) In vitro PCR amplification of pRRS-M.DraR1 plasmid: the pRRS-M.DraR1 plasmid is amplified in vitro by pfu high fidelity polymerase, an upstream amplification primer pRRS-F is shown in SEQ ID NO: 4, and a downstream amplification primer pRRS-PCR-R is shown in SEQ ID NO: 6. The amplified plasmid is purified and then used as a subsequent negative control study.

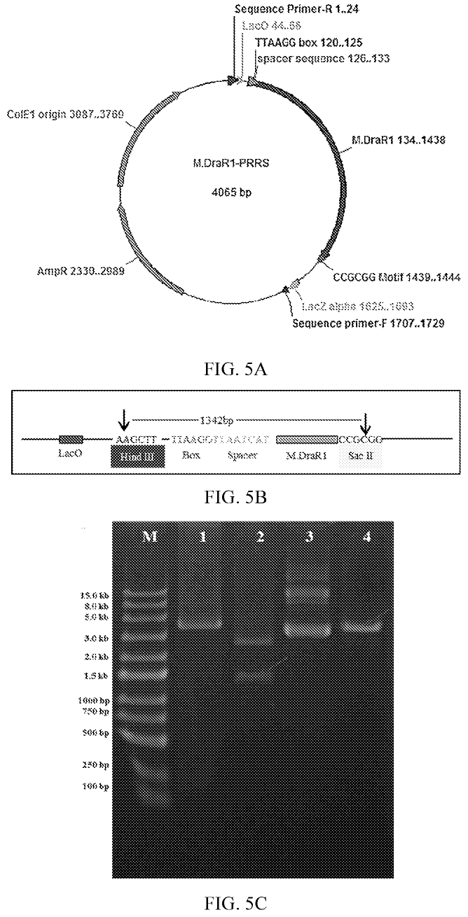

(4) M.DraR1 methyltransferase activity analysis: the above obtained plasmids are performed with digestion analysis by using restriction endonucleases of Hind III and Sac II; the “CCGCGG” sequence of the plasmid in (2) is methylated by M.DraR1 methylase, and thus it can avoid being digested by Sac II; the plasmid in (3) is not methylated, and thus it is digested. The results show that the DNA methyltransferase M.DraR1 can methylate the “CCGCGG” sequence in vivo (see FIG. 5B).

### Embodiment 3: DNA Methyltransferase M.DraR1 In Vitro Enzyme Activity Analysis

(1) Linearization treatment on pRRS-M.DraR1 plasmid: the above obtained plasmid is digested into linear segments by Hind III, and then is purified and recovered for later use.

(2) DNA methyltransferase M.DraR1 in vitro reaction system: 1-2 μg of each of the above treated plasmids is added into a reaction buffer containing 50-200 mM (millimole) KCl, 10-50 mM Tris-HCl (pH 7.5-8.0), 0.1 mM EDTA, 3-7 mM β-Me, and 20-100 μM (micromole) SAM, and moreover 1 μM of purified M.DraR1 protein is added, and then placed under suitable conditions for reaction.

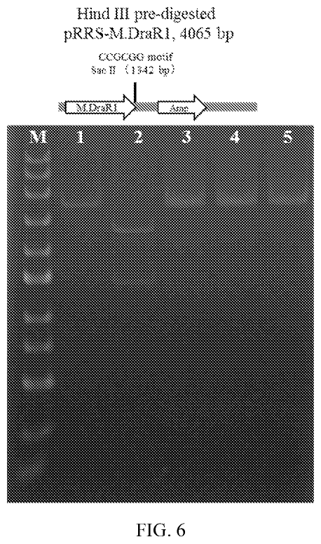

(3) Optimum enzyme activity temperature range of DNA methyltransferase M.DraR1: the above reaction system each is placed at 4-60° C. for 0.5-1 h, and after the purification reaction, plasmid DNA or bacteriophage DNA is digested by restriction endonuclease Sac II. The results further show that M.DraR1 methyltransferase can carry out methylated modification onto the “CCGCGG” conservative motif, and the optimum temperature range is 25-37° C. At 4° C., it still has weak methylation activity; and in the temperature range of 45-55° C., it still has methylation activity, but the activity is very weak (see FIG. 6).

The strain used in the illustrated embodiments of the invention is Deinococcus radiodurans R1, ATCC 13939, but according to the teaching and enlightenment of the invention, any artificially synthesized or other naturally contained proteins and their derivatives, such as those having homologous sequences and similar structures and functions to the DNA methyltransfer M.DraR1, are also within the protection scope of the invention.

1. A deoxyribonucleic acid (DNA) methyltransferase, having the amino acid sequence of SEQ ID NO: 1 and comprising: an adenosylmethionine (AdoMet) binding region having a “FxGxG” conservative sequence at positions 65 to 69 in the amino acid sequence of SEQ ID NO: 1, a target sequence recognition region and a catalytic region having a “TSPPY” conservative sequence of SEQ ID NO: 7, sequentially arranged in that order from N-terminal to C-terminal; and belonging to alpha-type DNA methyltransferase category;
wherein the target sequence recognition region of the DNA methyltransferase is used to recognize a DNA conservative sequence of 5′-CCGCGG-3′, and the catalytic region of the DNA methyltransferase is used to methylation modify N4 site of second cytosine (C) thereof to generate a N4-methylcytosine (4mC) type modified base;
wherein a method for recognition specificity of the DNA methyltransferase from Deinococcus radiodurans, comprises:
linking a streptomycin resistance gene segment with an upstream segment and a downstream segment of the amino acid sequence of the DNA methyltransferase into a whole segment in vitro to obtain a triple-segment product;
transforming the triple-segment product into a competent cell of Deinococcus radiodurans;
screening mutant strains by culturing the competent cell of Deinococcus radiodurans;
obtaining a gene knockout strain of the DNA methyltransferase based on the mutant strains;
extracting genomes of a wild-type Deinococcus radiodurans R1 (DraR1) and the gene knockout strain of the DNA methyltransferase; and
performing sequencing analysis on the extracted genomes of the wild-type DraR1 and the gene knockout strain, and obtaining a sequencing analysis result that the DNA methyltransferase is capable of recognizing the DNA conservative sequence of 5′-CCGCGG-3′, and methylation modifying N4 site of second cytosine (C) thereof to generate the 4mC type modified base.

2. The DNA methyltransferase as claimed in claim 1, wherein a reaction buffer for the DNA methyltransferase contains 50-200 millimoles (mM) potassium chloride (KCl), 10-50 mM trimethylolaminomethane hydrochloride (Tris-HCl) with pH7.5-8.0, 0.1 mM ethylenediaminetetraacetic acid (EDTA), 3-7 mM 3-Mercaptoethanol (β-Me), and 20-100 micromoles (μM) S-adenosyl methionine (SAM).

3. The DNA methyltransferase as claimed in claim 1, wherein an enzyme activity temperature range of the DNA methyltransferase is 4-60 Celsius degrees (° C.).

4. The DNA methyltransferase as claimed in claim 3, wherein an optimum enzyme activity temperature range of the DNA methyltransferase is 25-37° C.

5. The DNA methyltransferase as claimed in claim 1, wherein the target sequence recognition region is the TRD sequence.


SEQUENCE LISTING (6 sequences)

SEQ ID NO: 1 - protein, 434 aa, Unknown
  Methyl donor AdoMet binding region, FxGxG conservative sequence, Target sequence recognition region TRD, Methylation catalytic region, TSPPY conservative sequence (30)...(140),  (65)...(69), (145)...(250),(250)...(400), (255)...(259): Deinococcus radiodurans DNA methyltransferase amino acid sequence
MTQPLLFDLPTPRPTYRDTAFASNKTLAMHRWVNWIAGFSSEFVQHALELHLPDPNPEQVVLDPFGGVGTTPITAFLRGHSVVSYDINPFPLLVQRAKLRAIQDVTPAEFAQQIEAFTAHMATGGVPKSKVPQGFTSRTPFYSEKVLVKVLHVWDFINEVADEDLRDLFQVAFGATMVSYSNYSYEPSLGSRAAAGKPDIEDADVAQVMRDKLLEMHADLLGVQGIKLGGQTAQVYQGSFMRSELPDSSVDLMVTSPPYLNNYHYLRNTRPHLYWLGYATSPKDLRYLELDNYGKYWQTVRDAKYQTSLIFDSPWLQDLVNQLAGVQSDRGVYGGQGWANYATEYFNDTYRFLQKTQAVLRPGAKALIVVGNSIVKGTNLPIDEVFTHIAQHLGFSGHDIHMVRDSRIGSSIVGTGLRSEGKGRLYEAVVELTR

SEQ ID NO: 2 - DNA, 48 bp, Artificial
  Synthetic primer pRRS-M.DraR1-F
tttcctgcaggttaaggttaatcatatgacgcaacctcttctctttga

SEQ ID NO: 3 - DNA, 55 bp, Artificial
  Synthetic primer pRRS-M.DraR1-R
tttggatccccgcggttagtggtggtggtggtggtgcctggtcagttcaaccacg

SEQ ID NO: 4 - DNA, 24 bp, Artificial
  Synthetic sequencing primer pRRS-F
accccaggctttacactttatgct

SEQ ID NO: 5 - DNA, 23 bp, Artificial
  Synthetic sequencing primer pRRS-R
gcacagatgcgtaaggagaaaat

SEQ ID NO: 6 - DNA, 24 bp, Artificial
  Synthetic primer pRRS-PCR-R
agcataaagtgtaaagcctggggt